(19) United States
(12) Patent Application Publication
(10) Pub. No.: US 2022/0211347 A1
(43) Pub. Date: Jul. 7, 2022

(54) METHOD AND SYSTEM FOR AUTOMATICALLY DETECTING AN APEX POINT IN APICAL ULTRASOUND IMAGE VIEWS TO PROVIDE A FORESHORTENING WARNING

(71) Applicant: GE Precision Healthcare LLC, Wauwatosa, WI (US)

(72) Inventor: Kristin Sarah McLeod, Oslo (NO)

(21) Appl. No.: 17/140,883

(22) Filed: Jan. 4, 2021

(51) Int. Cl. A61B 8/00; G06T 7/246; G06T 11/00; A61B 8/08

(52) U.S. Cl. CPC A61B 8/463; G06T 7/248; G06T 11/001; A61B 8/0883; G06T 2207/30048; G06T 2207/10132; A61B 8/5276

(57) ABSTRACT

A system and method for automatically detecting an apex point in apical ultrasound image views to provide a foreshortening warning is provided. The method may include acquiring, by an ultrasound system, apical ultrasound image views over a time period. The method may include automatically detecting, by at least one processor, an apical point in the apical ultrasound image views. The method may include determining, by the at least one processor, an amount of movement of the apical point in the apical ultrasound image views over the time period. The method may include causing, by the at least one processor, a display system to present the apical ultrasound image views with a foreshortening warning when the amount of movement of the apical point in the apical ultrasound image views over the time period exceeds a threshold.

### FIELD

Certain embodiments relate to ultrasound imaging. More specifically, certain embodiments relate to a method and system for providing a foreshortening warning in apical ultrasound image views by automatically detecting an apex point in the apical ultrasound image views.

### BACKGROUND

Ultrasound imaging is a medical imaging technique for imaging organs and soft tissues in a human body. Ultrasound imaging uses real time, non-invasive high frequency sound waves to produce a series of two-dimensional (2D) and/or three-dimensional (3D) images.

During an echocardiogram ultrasound imaging procedure, an ultrasound operator may acquire apical views of the heart over a time period, such as one or more cardiac cycles. The apical ultrasound image views may be used to perform various measurements, such as an ejection fraction measurement, among other things. However, foreshortening of apical ultrasound image views may cause inaccurate measurements that may lead to an incorrect diagnosis.

Foreshortening of apical views is a common problem in echocardiography that typically results in an abnormally thick false apex and a shortened left ventricular (LV) long axis. Foreshortening refers to a situation where the ultrasound plane does not cut through the true apex of the left ventricle but transects above and anterior of the true apex. Foreshortened apical ultrasound image views provide a geometric distortion of the image of the left ventricle, making the apex look “rounded” instead of the normal “bullet” shape. As a result, the long axis of the left ventricle appears shorter and the false apex is thicker and apparently hyper-contractile resulting in an overestimation of both global and regional LV function and an underestimation of LV volume and length. In addition, apical thrombus or infarction can be missed in a foreshortened view.

Currently, foreshortening may be manually determined by an experienced ultrasound operator viewing the acquired apical ultrasound views. For less experienced operators or if the experienced ultrasound operator does not recognize the foreshortening, the determination may not be made until measurements are performed, if at all. For example, an ultrasound operator may determine that foreshortening is present based on unusual measurements, such as an ejection fraction measurement that appears overly low.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for automatically detecting an apex point in apical ultrasound image views to provide a foreshortening warning, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to automatically detect an apex point in apical ultrasound image views to provide a foreshortening warning, in accordance with various embodiments.

FIG. 2 is a display of an exemplary electrocardiogram (ECG) and an exemplary apical ultrasound image view having an apical point marker identifying an apex of a heart, in accordance with various embodiments.

FIG. 3 is a display of an exemplary ECG and an exemplary apical ultrasound image view having a first apical point marker identifying an apex of a heart of the current apical ultrasound image view and a second apical point marker identifying a location of the apex in an apical ultrasound image view acquired at a different time, in accordance with various embodiments.

FIG. 4 is a display of an exemplary foreshortening warning message, an exemplary ECG, and an exemplary apical ultrasound image view having a first apical point marker identifying an apex of a heart of the current apical ultrasound image view, a second apical point marker identifying a location of the apex in an apical ultrasound image view acquired at a different time, and a distance marker illustrating a distance between the first and second apical point markers, in accordance with various embodiments.

FIG. 5 is a flow chart illustrating exemplary steps that may be utilized for automatically detecting an apex point in apical ultrasound image views to provide a foreshortening warning, in accordance with various embodiments.

### DETAILED DESCRIPTION

Certain embodiments may be found in a method and system for automatically detecting an apex point in apical ultrasound image views to provide a foreshortening warning. Aspects of the present disclosure have the technical effect of presenting the apex point for the user on apical ultrasound image views as a visual support in addition to a foreshortening warning message. Various embodiments have the technical effect of automatically detecting the apical point using, for example, artificial intelligence and/or other image processing techniques. Certain embodiments have the technical effect of providing foreshortening warnings by either, for example, comparing the apical points at end diastole and end systole and showing both apical points overlaid on the apical ultrasound image view to visualize the distance, or by plotting on all frames so that the user can see the motion over the cycle. Aspects of the present disclosure have the technical effect of presenting the automatically detected apex point on apical ultrasound image views to demonstrate potential foreshortening. Various embodiments have the technical effect of addressing the challenge of providing feedback to the user live or post-acquisition of an automatically detected foreshortened acquisition, without crowding or cluttering the user interface. Certain embodiments have the technical effect of providing immediate user feedback of foreshortened views.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, Contrast Enhanced Ultrasound (CEUS), and/or sub-modes of B-mode and/or CF such as Harmonic Imaging, Shear Wave Elasticity Imaging (SWEI), Strain Elastography, TVI, PDI, B-flow, MVI, UGAP, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphic Processing Unit (GPU), DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

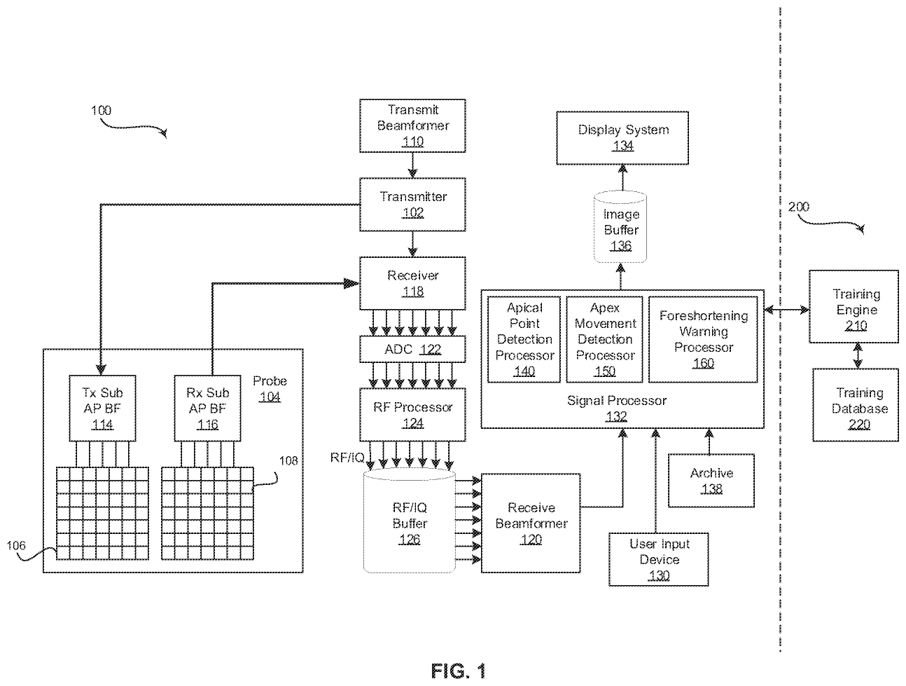

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to automatically detect an apex point in apical ultrasound image views to provide a foreshortening warning, in accordance with various embodiments. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as the heart, a blood vessel, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise an apical point detection processor 140, an apex movement detection processor 150, and a foreshortening warning processor 160. The signal processor 132 may be capable of receiving input information from a user input device 130 and/or archive 138, generating an output displayable by a display system 134, and manipulating the output in response to input information from a user input device 130, among other things. The signal processor 132, apical point detection processor 140, apex movement detection processor 150, and foreshortening warning processor 160 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include an apical point detection processor 140 that comprises suitable logic, circuitry, interfaces and/or code that may be operable to analyze acquired apical ultrasound image views, such as an apical two chamber (2CH) or four chamber (4CH) view, to automatically identify an apical point in the apical ultrasound image views. The apical point detection processor 140 may include image analysis algorithms, artificial intelligence algorithms, one or more deep neural networks (e.g., a convolutional neural network) and/or may utilize any suitable form of image analysis techniques or machine learning processing functionality configured to analyze acquired apical ultrasound image views to automatically identify an apical point in the apical ultrasound image views. The apical point detection processor 140 may be configured to provide the locations of the apical points in the apical ultrasound image views to the apex movement detection processor 150 and/or the foreshortening warning processor 160. The apical point detection processor 140 may additionally and/or alternatively store the apical point locations at archive 138 and/or any suitable data storage medium.

The apical point detection processor 140 may comprise suitable logic, circuitry, interfaces and/or code that may be operable to analyze acquired apical ultrasound image views to identify an apical point. In various embodiments, the apical point detection processor 140 may be provided as a deep neural network that may be made up of, for example, an input layer, an output layer, and one or more hidden layers in between the input and output layers. Each of the layers may be made up of a plurality of processing nodes that may be referred to as neurons. For example, the apical point detection processor 140 may include an input layer having a neuron for each pixel or a group of pixels from a scan plane of an apical view of the heart. The output layer may have a neuron corresponding to an apical point. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The processing performed by the apical point detection processor 140 deep neural network (e.g., convolutional neural network) may identify an apical point in ultrasound image data with a high degree of probability.

The signal processor 132 may include apex movement detection processor 150 that comprises suitable logic, circuitry, interfaces and/or code that may be operable to determine whether the apical point identified by the apical point detection processor 140 is moving in the apical ultrasound image views over a time period (e.g., a cardiac cycle). For example, the apex movement detection processor 150 may be configured to determine distances between the apical points in successive frames and/or pre-defined frames (e.g., between current view and end diastole view, between current view and end systole view, between end diastole view and end systole view, or the like). The apex movement detection processor 150 may be configured to determine whether the determined distance exceeds a threshold. The threshold may correspond to no and/or negligible apex movement. The apex movement detection processor 150 may be configured to provide the determined amount of movement to the foreshortening warning processor 160. The apex movement detection processor 150 may additionally and/or alternatively store the determined amount of movement at archive 138 and/or any suitable data storage medium.

The signal processor 132 may include a foreshortening warning processor 160 that comprises suitable logic, circuitry, interfaces and/or code that may be operable to display indications of whether foreshortening is present in the acquired apical ultrasound image views. The foreshortening indications may comprise one or more of apical marker(s), a movement distance indicator, a foreshortening message, and/or any suitable warning indication. For example, the foreshortening warning processor 160 may display an apical point marker if foreshortening is present. The apical point marker may be overlaid at the apex or may be colorized pixels of the apex, for example. The apical point marker may be a shape, such as a dot, circle, star, square, box, arrow, and/or any suitable shape. The foreshortening warning processor 160 may be configured to position the apical point marker at locations identified by the apical point detection processor 140. The apical point marker may be color-coded to define an amount of movement as indicated by the apex movement detection processor 150. For example, a green apical marker may correspond with no or negligible apex movement, an orange apical marker may correspond with some apex movement, and a red apical marker may correspond with a large amount of apex movement. In various embodiments, an apical marker may not be presented by the foreshortening warning processor 160 when no or negligible movement is detected by the apex movement detection processor 150.

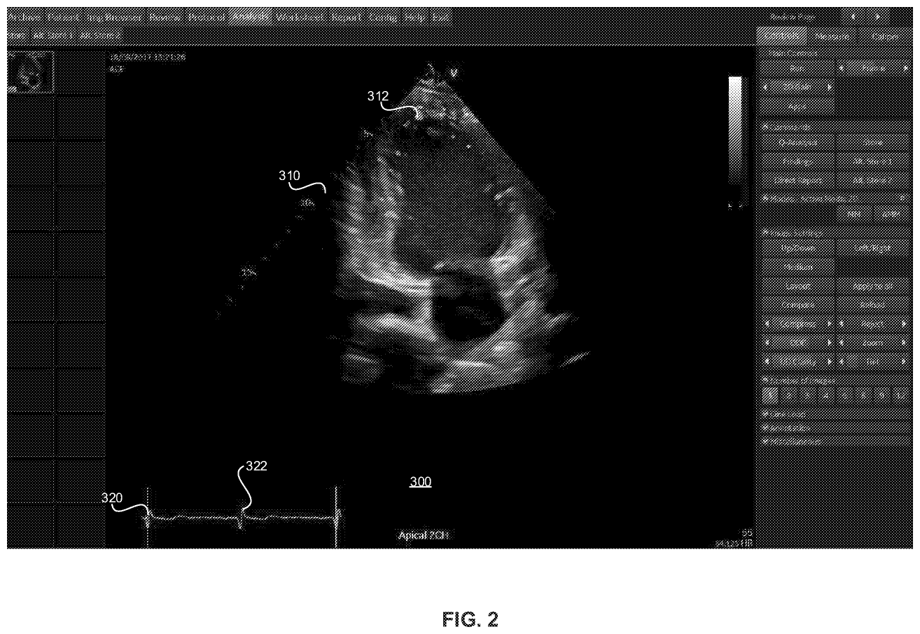

FIG. 2 is a display 300 of an exemplary electrocardiogram (ECG) 320 and an exemplary apical ultrasound image view 310 having an apical point marker 312 identifying an apex of a heart, in accordance with various embodiments. Referring to FIG. 2, the display 300 may comprise an apical ultrasound image view 310 and an ECG 320. The foreshortening warning processor 160 may be configured to colorize pixels of the apex and/or overlay an apical point marker 312 on the apical ultrasound image view 310 based on the apex location detected by the apical point detection processor 140. The apical point marker 312 may be color-coded based on an amount of movement indicated by the apex movement detection processor 150. Alternatively, an amount of movement may be distinguished based on different apical point marker shapes, labels, shading, or the like. The foreshortening warning processor 160 may be configured to identify the temporal location of the displayed apical ultrasound image view 310. For example, the foreshortening warning processor 160 may be configured to colorize pixels or overlay an ECG marker 322 in the ECG 320 to identify the location of the apical ultrasound image view 310 in the cardiac cycle.

Referring again to FIG. 1, the foreshortening warning processor 160 may be configured to display multiple apical point markers in an apical ultrasound image view. For example, a first apical point marker may correspond with a location of the apex in the current apical ultrasound image view and a second apical point marker may correspond with an apex location of a previous frame and/or other pre-defined frame (e.g., end of diastole view, end of systole view, and/or the like). In certain embodiments, the foreshortening warning processor 160 may be configured to display a movement distance indicator. For example, the foreshortening warning processor 160 may display a line connecting multiple apical point markers, a distance measurement between apical point markers, and/or any suitable movement distance indication.

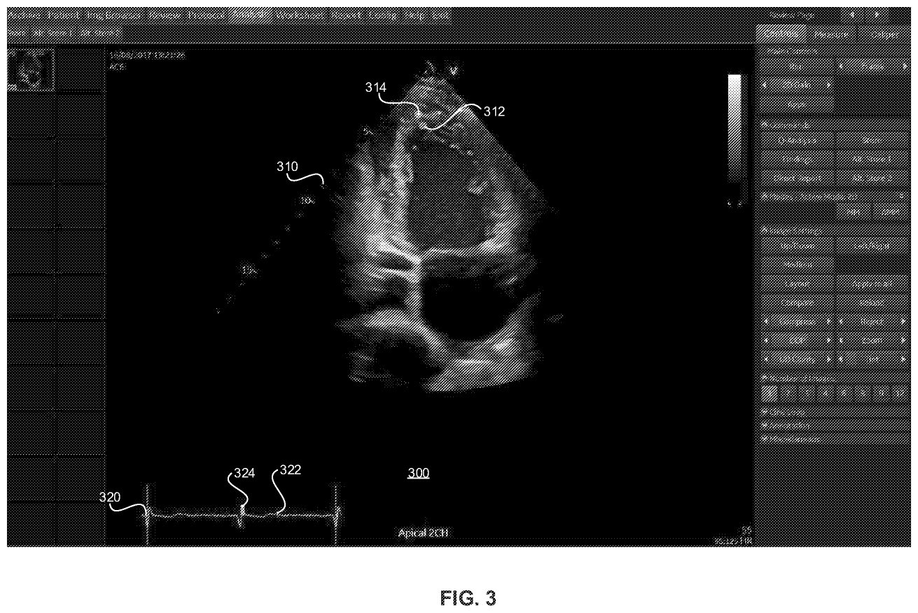

FIG. 3 is a display 300 of an exemplary ECG 320 and an exemplary apical ultrasound image view 310 having a first apical point marker 312 identifying an apex of a heart of the current apical ultrasound image view 310 and a second apical point marker 314 identifying a location of the apex in an apical ultrasound image view acquired at a different time, in accordance with various embodiments. Referring to FIG. 3, the display 300 may comprise an apical ultrasound image view 310 and an ECG 320. The foreshortening warning processor 160 may be configured to colorize pixels of the apex and/or overlay apical point markers 312, 314 on the apical ultrasound image view 310 based on the apex location in the current view 310 and a previous or pre-defined view (e.g., end of diastole view, end of systole view, or the like) as detected by the apical point detection processor 140. The apical point markers 312, 314 may be color-coded to distinguish between the different markers 312, 314 and/or based on an amount of movement indicated by the apex movement detection processor 150. Alternatively, the different markers and/or amount of movement may be distinguished based on different apical point marker shapes, labels, shading, or the like. The foreshortening warning processor 160 may be configured to identify the temporal locations of the displayed apical ultrasound image view 310 and the image view corresponding with the second apical point marker 314. For example, the foreshortening warning processor 160 may be configured to colorize pixels or overlay ECG markers 322, 324 in the ECG 320 to identify the location 322 of the apical ultrasound image view 310 and the location 324 of the previous apical ultrasound image view in the cardiac cycle.

Referring again to FIG. 1, the foreshortening warning processor 160 may be configured to present a foreshortening message. The foreshortening message may be in addition to or an alternative to the apical point marker(s) and/or the movement distance indication. The foreshortening message may be a visual warning, audio warning, and/or physical warning. The visual warning may be a visual message presented at the display system 134 or any suitable visual warning. For example, the visual warning may be a message stating, “Warning: foreshortened view detected.” The audible warning may be an alarm, message, or any suitable audible feedback. The physical warning may include causing the probe 104 to vibrate, or any suitable physical warning.

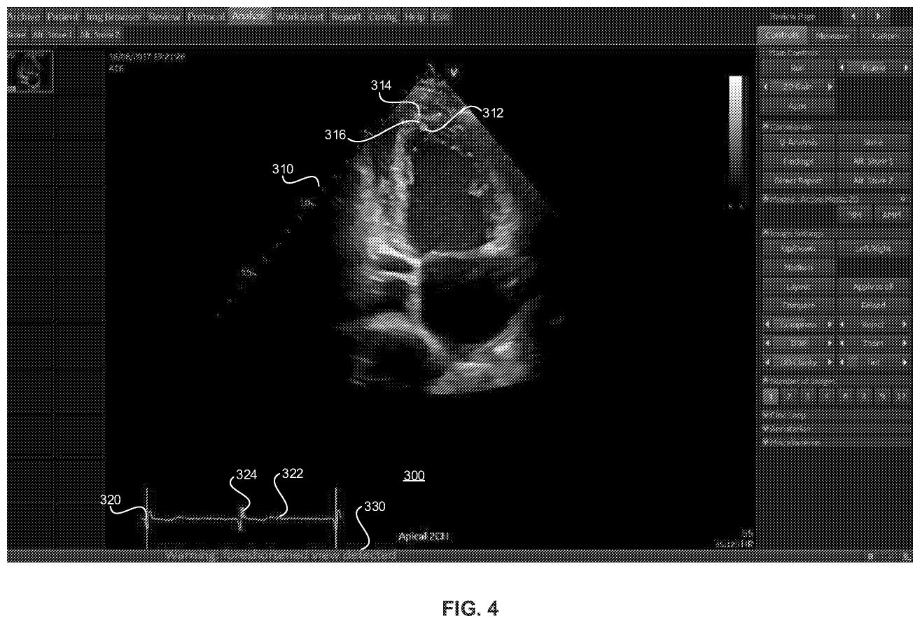

FIG. 4 is a display 300 of an exemplary foreshortening warning message 330, an exemplary ECG 320, and an exemplary apical ultrasound image view 310 having a first apical point marker 312 identifying an apex of a heart of the current apical ultrasound image view 310, a second apical point marker 314 identifying a location of the apex in an apical ultrasound image view acquired at a different time, and a distance marker 316 illustrating a distance between the first 312 and second 314 apical point markers, in accordance with various embodiments. Referring to FIG. 4, the display 300 may comprise an apical ultrasound image view 310, an ECG 320, and a foreshortening warning message 330. The foreshortening warning processor 160 may be configured to colorize pixels of the apex and/or overlay apical point markers 312, 314 on the apical ultrasound image view 310 based on the apex location in the current view 310 and a previous or pre-defined view (e.g., end of diastole view, end of systole view, or the like) as detected by the apical point detection processor 140. The apical point markers 312, 314 may be color-coded to distinguish between the different markers 312, 314 and/or based on an amount of movement indicated by the apex movement detection processor 150. Alternatively, the different markers and/or amount of movement may be distinguished based on different apical point marker shapes, labels, shading, or the like. The foreshortening warning processor 160 may be configured to identify the temporal locations of the displayed apical ultrasound image view 310 and the image view corresponding with the second apical point marker 314. For example, the foreshortening warning processor 160 may be configured to colorize pixels or overlay ECG markers 322, 324 in the ECG 320 to identify the location 322 of the apical ultrasound image view 310 and the location 324 of the previous apical ultrasound image view in the cardiac cycle. The foreshortening warning processor 160 may be configured to present a distance marker 316 (also referred to as a movement distance indicator) illustrating an amount of movement. The distance marker 316 may include a line 316 connecting the apical point markers 312, 314, labels, and/or any suitable movement distance indicator. The distance marker may be color-coded, shaded, or the like to provide additional information related to the amount of movement. For example, different colors, shading, or the like may correspond with different amounts of movement. The foreshortening warning processor 160 may be configured to provide a foreshortening message, such as a visual warning 330, an audio warning, and/or a physical warning.

Referring again to FIG. 1, the display system 134 may be any device capable of communicating visual information to a user. For example, a display system 134 may include a liquid crystal display, a light emitting diode display, and/or any suitable display or displays. The display system 134 can be operable to present apical ultrasound image views 310, an ECG 320, visual foreshortening warnings 330, and/or any suitable information. For example, the apical ultrasound image views 310, ECG 320, and visual foreshortening warnings 330 presented at the display system 134 may include one or more of apical marker(s) 312, 314, a movement distance indicator 316, a foreshortening message 330, ECG marker(s) 322, 324, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores ultrasound image data, apical point locations, apical point movement information, foreshortening warnings, apical point detection instructions, apex movement detection instructions, and foreshortening warning instructions, for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

Still referring to FIG. 1, the training system 200 may comprise a training engine 210 and a training database 220. The training engine 160 may comprise suitable logic, circuitry, interfaces and/or code that may be operable to train the neurons of the deep neural network(s) (e.g., artificial intelligence model(s)) inferenced (i.e., deployed) by the apical point detection processor 140. For example, the artificial intelligence model inferenced by the apical point detection processor 140 may be trained to automatically identify anatomical features (e.g., an apex) in ultrasound images (e.g., apical ultrasound image views 310). As an example, the training engine 210 may train the deep neural networks deployed by the apical point detection processor 140 using database(s) 220 of classified two chamber (2CH) and four chamber (4CH) apical ultrasound image views. The ultrasound images may include ultrasound images of a particular anatomical feature, such as apical ultrasound image views 310 having an apex, or any suitable ultrasound images and features.

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the ultrasound system 100 as shown in FIG. 1. Additionally and/or alternatively, components or all of the training system 200 may be integrated with the ultrasound system 100 in various forms.

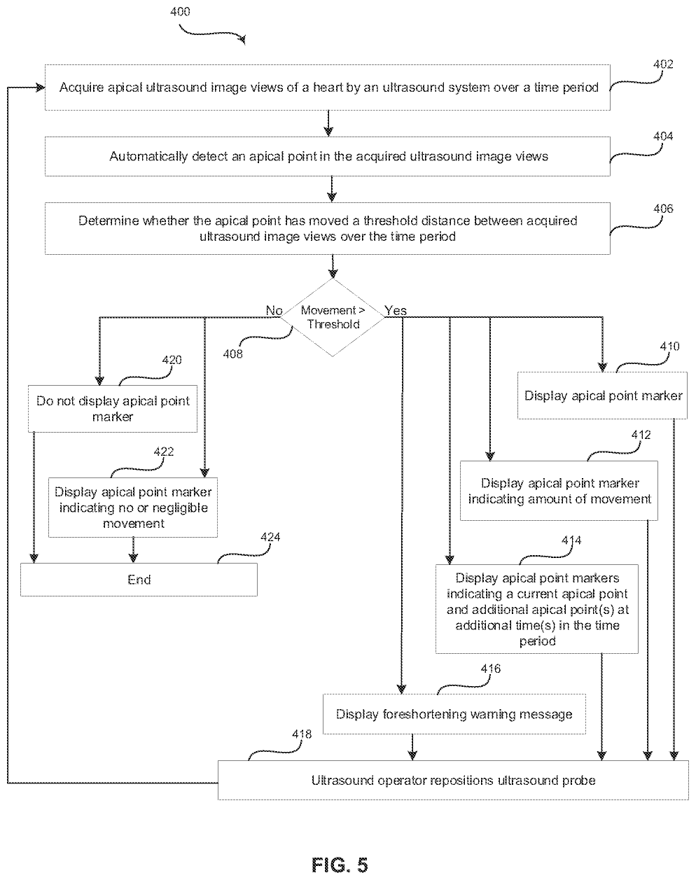

FIG. 5 is a flow chart 400 illustrating exemplary steps 402-424 that may be utilized for automatically detecting an apex point in apical ultrasound image views 310 to provide a foreshortening warning 312, 314, 316, 330, in accordance with various embodiments. Referring to FIG. 5, there is shown a flow chart 400 comprising exemplary steps 402 through 424. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 402, an ultrasound system 100 acquires apical ultrasound image views 310 of a heart over a time period. For example, the ultrasound system 100 may acquire apical ultrasound image views 310, such as apical two chamber (2CH) or four chamber (4CH) views, with an ultrasound probe 104 positioned at a scan position over a heart during a cardiac cycle. In various embodiments, the ultrasound system 100 may receive additional information, such as an ECG 320 and/or any suitable information.

At step 404, a signal processor 132 of the ultrasound system 100 automatically detects an apical point in the acquired ultrasound image views 310. For example, an apical point detection processor 140 of the signal processor 132 may be configured to analyze the acquired apical ultrasound image views 310 to automatically identify an apical point in the apical ultrasound image views 310. The apical point detection processor 140 may include image analysis algorithms, artificial intelligence algorithms, one or more deep neural networks (e.g., a convolutional neural network) and/or may utilize any suitable form of image analysis techniques or machine learning processing functionality configured to analyze acquired apical ultrasound image views 310 to automatically identify the apical point in the apical ultrasound image views 310.

At step 406, the signal processor 132 of the ultrasound system 100 may determine whether the apical point has moved a threshold distance between acquired ultrasound image views over the time period. For example, the apex movement detection processor 150 of the signal processor 132 may be configured to determine whether the apical point identified by the apical point detection processor 140 at step 404 is moving in the apical ultrasound image views 310 over a cardiac cycle. The apex movement detection processor 150 may be configured to determine distances between the apical points in successive frames and/or pre-defined frames (e.g., between current view and end diastole view, between current view and end systole view, between end diastole view and end systole view, or the like). The apex movement detection processor 150 may be configured to determine whether the determined distance exceeds a threshold, which may correspond to no and/or negligible apex movement. The threshold may be user-defined or a default value. In various embodiments, the threshold may include multiple thresholds each corresponding to a different amount of movement (e.g., no movement, negligible movement, medium movement, large movement, and the like).

At step 408, the process 400 may proceed to one or more of steps 410 through 416 if the signal processor 132 of the ultrasound system 100 determines the apical point has moved more than the threshold distance between acquired apical ultrasound image views 310 at step 406. Additionally and/or alternatively, the process 400 may proceed to one of steps 420 or 422 if the signal processor 132 of the ultrasound system 100 determines the apical point has not moved more than the threshold distance between acquired apical ultrasound image views 310 at step 406.

At step 410, the signal processor 132 of the ultrasound system 100 may display an apical point marker 312. For example, the foreshortening warning processor 160 of the signal processor 132 may display indications of whether foreshortening is present in the acquired apical ultrasound image views. The foreshortening indications may comprise an apical point marker 312 if foreshortening is present. The apical point marker 312 may be overlaid at the apex or may be colorized pixels of the apex, for example. The apical point marker may be a shape, such as a dot, circle, star, square, box, arrow, and/or any suitable shape. The foreshortening warning processor 160 may be configured to position the apical point marker 312 at locations identified by the apical point detection processor 140. The foreshortening warning processor 160 may be configured to identify the temporal location of the displayed apical ultrasound image view 310. For example, the foreshortening warning processor 160 may be configured to colorize pixels or overlay an ECG marker 322 in the ECG 320 to identify the location of the apical ultrasound image view 310 in the cardiac cycle.

At step 412, the signal processor 132 of the ultrasound system 100 may display an apical point marker 312 indicating an amount of movement. For example, the foreshortening warning processor 160 of the signal processor 132 may display apical point markers 312 that are color-coded, shaded, shaped, labeled, or the like to indicate an amount of movement as determined by the apex movement detection processor 150. As an example, an orange apical marker may correspond with some apex movement and a red apical marker may correspond with a large amount of apex movement. As another example, the apical point marker 312 may be labeled with an amount of movement. As a further example, different shapes of apical point markers 312 may be used based on the amount of movement as determined by the apex movement detection processor 150.

At step 414, the signal processor 132 of the ultrasound system 100 may display apical point markers 312, 314 indicating a current apical point and additional apical point(s) at additional time(s) in the time period. For example, the foreshortening warning processor 160 of the signal processor 132 may be configured to display multiple apical point markers 312, 314 in an apical ultrasound image view 310. As an example, a first apical point marker 312 may correspond with a location of the apex in the current apical ultrasound image view 310 and a second apical point marker 314 may correspond with an apex location of a previous frame and/or other pre-defined frame (e.g., end of diastole view, end of systole view, and/or the like). In an exemplary embodiment, the foreshortening warning processor 160 may be further configured to display a movement distance indicator 316. For example, the foreshortening warning processor 160 may display a line 316 connecting multiple apical point markers 312, 314, a distance measurement between apical point markers, and/or any suitable movement distance indication. The foreshortening warning processor 160 may be configured to identify the temporal locations of the displayed apical ultrasound image view 310 and the image view corresponding with the second apical point marker 314. For example, the foreshortening warning processor 160 may be configured to colorize pixels or overlay ECG markers 322, 324 in the ECG 320 to identify the location 322 of the apical ultrasound image view 310 and the location 324 of the previous apical ultrasound image view in the cardiac cycle.

At step 416, the signal processor 132 of the ultrasound system 100 may display a foreshortening warning message 330. For example, the foreshortening warning processor 160 of the signal processor 132 may be configured to present a foreshortening message 330. The foreshortening message may be in addition to or an alternative to the apical point marker(s) 312, 314 and/or the movement distance indication 316 described above with respect to steps 410-414. The foreshortening message 330 may be a visual warning 330, audio warning, and/or physical warning. The visual warning 330 may be a visual message presented at the display system 134 or any suitable visual warning. For example, the visual warning 330 may be a message stating, “Warning: foreshortened view detected.” The audible warning may be an alarm, message, or any suitable audible feedback. The physical warning may include causing the probe 104 to vibrate, or any suitable physical warning

At step 418, an ultrasound operator may reposition the ultrasound probe 104 of the ultrasound system 100. For example, the ultrasound operator may move and/or rotate the ultrasound probe 104 of the ultrasound system in response to the foreshortening warning 312, 314, 316, 330 provided by the foreshortening warning processor 160 of the signal processor 132 of the ultrasound system 100. The process 400 returns to step 402 and repeats steps 402-418 until the signal processor 132 of the ultrasound system 100 determines the apical point has not moved more than the threshold distance between acquired apical ultrasound image views 310 at step 406, at which time, the process 400 proceeds to step 420 or 422.

At step 420, the signal processor 132 of the ultrasound system 100 may not display the apical point marker 312. For example, the foreshortening warning processor 160 of the signal processor 132 may be configured to refrain from displaying the apical point marker 312 if the signal processor 132 of the ultrasound system 100 determines the apical point has not moved more than the threshold distance between acquired apical ultrasound image views 310 at step 406.

At step 422, the signal processor 132 of the ultrasound system 100 may display an apical point marker 312 indicating no or negligible movement. For example, the foreshortening warning processor 160 of the signal processor 132 may display apical point markers 312 that are color-coded, shaded, shaped, labeled, or the like to indicate an amount of movement as determined by the apex movement detection processor 150. As an example, a green apical marker may correspond with no or negligible apex movement. As another example, the apical point marker 312 may be labeled with an amount of movement. As a further example, different shapes of apical point markers 312 may be used based on the amount of movement as determined by the apex movement detection processor 150.

At step 424, the process 300 may end when acquisition of apical ultrasound image views 310 having no or negligible movement is complete. The ultrasound operator and/or signal processor 132 of the ultrasound system 100 may use the apical ultrasound image views 310 not having foreshortening to perform measurements (e.g., an ejection fraction measurement), diagnosis, or the like.

Aspects of the present disclosure provide a method 400 and system 100 for automatically detecting an apex point in apical ultrasound image views 310 to provide a foreshortening warning 312, 314, 316, 330. In accordance with various embodiments, the method 400 may comprise acquiring 402, by an ultrasound system 100, apical ultrasound image views 310 over a time period. The method 400 may comprise automatically detecting 404, by at least one processor 132, 140, an apical point in the apical ultrasound image views 310. The method 400 may comprise determining 406, 408, by the at least one processor 132, 150, an amount of movement of the apical point in the apical ultrasound image views 310 over the time period. The method 400 may comprise causing 410-416, by the at least one processor 132, 160, a display system 134 to present the apical ultrasound image views 310 with a foreshortening warning 312, 314, 316, 300 when the amount of movement of the apical point in the apical ultrasound image views 310 over the time period exceeds a threshold.

In an exemplary embodiment, the foreshortening warning 312, 314, 316, 330 may comprise at least one apical point marker 312, 314 presented at the automatically detected apical point in the apical ultrasound image views 310. In a representative embodiment, the at least one apical point marker 312, 314 may be one apical point marker 312 indicating the amount of movement based on color coding in one of a plurality of colors, where each of the plurality of colors corresponds with a different amount of movement. The one apical point marker 312 may additionally and/or alternatively indicate the amount of movement based on shading in one of a plurality of shades, where each of the plurality of shades corresponds to the different amount of movement. The one apical point marker 312 may additionally and/or alternatively indicate the amount of movement based on marker shape selection in one of a plurality of marker shapes, where each of the plurality of marker shapes corresponds to the different amount of movement. The one apical point marker 312 may additionally and/or alternatively indicate the amount of movement based on a label corresponding with the one apical point marker 312, where the label identifies the amount of movement. In certain embodiments, the at least one apical point marker 312, 314 may comprise a first apical point marker 312 corresponding with a currently displayed apical ultrasound image view 310 and a second apical point marker 314 corresponding with a previous apical ultrasound image view in the time period. In various embodiments, the foreshortening warning 312, 314, 316, 330 may comprise a distance marker 316 corresponding to a distance between the first apical point marker 312 and the second apical point marker 314. In an exemplary embodiment, the at least one apical point marker 312, 314 is one of colorized pixels 312, 314 of the apical ultrasound image views 310 or a marker 312, 314 overlaid on the apical ultrasound image views 310. In a representative embodiment, the foreshortening warning 312, 314, 316, 330 may comprise a foreshortening warning message 330. In certain embodiments, the method 400 may comprise causing 422, by the at least one processor 132, 160, the display system 134 to present the apical ultrasound image views 310 with at least one apical point marker 312, 314 provided at the automatically detected apical point in the apical ultrasound image views 310 when the amount of movement of the apical point in the apical ultrasound image views 310 over the time period does not exceed the threshold. The at least one apical point marker 312, 314 may indicate one or both of no movement or negligible movement. The method 400 may comprise causing 420, by the at least one processor 132, 160, the display system 134 to present the apical ultrasound image views 310 without the at least one apical point marker 312, 314 when the amount of movement of the apical point in the apical ultrasound image views 310 over the time period does not exceed the threshold.

Various embodiments provide a system 100 for automatically detecting an apex point in apical ultrasound image views 310 to provide a foreshortening warning 312, 314, 316, 330. The system 100 may comprise an ultrasound system 100, at least one processor 132, 140, 150, 160, and a display system 134. The ultrasound system 100 may be configured to acquire apical ultrasound image views 310 over a time period. The at least one processor 132, 140 may be configured to automatically detect an apical point in the apical ultrasound image views 310. The at least one processor 132, 150 may be configured to determine an amount of movement of the apical point in the apical ultrasound image views 310 over the time period. The display system 134 may be configured to present the apical ultrasound image views 310 with a foreshortening warning 312, 314, 316, 330 when the amount of movement of the apical point in the apical ultrasound image views 310 over the time period exceeds a threshold.

In a representative embodiment, the foreshortening warning 312, 314, 316, 330 may comprise an apical point marker 312 presented at the automatically detected apical point in the apical ultrasound image views 310. The apical point marker 312 may indicate the amount of movement based on color coding in one of a plurality of colors, where each of the plurality of colors corresponds with a different amount of movement. The apical point marker 312 may additionally and/or alternatively indicate the amount of movement based on shading in one of a plurality of shades, where each of the plurality of shades corresponds to the different amount of movement. The apical point marker 312 may additionally and/or alternatively indicate the amount of movement based on marker shape selection in one of a plurality of marker shapes, where each of the plurality of marker shapes corresponds to the different amount of movement. The apical point marker 312 may additionally and/or alternatively indicate the amount of movement based on a label corresponding with the apical point marker 312, where the label identifies the amount of movement. In certain embodiments, the foreshortening warning 312, 314, 316, 330 may comprise a first apical point marker 312 corresponding with a currently displayed apical ultrasound image view 310 and a second apical point marker 314 corresponding with a previous apical ultrasound image view in the time period. In various embodiments, the foreshortening warning 312, 314, 316, 330 may comprise a distance marker 316 corresponding to a distance between the first apical point marker 312 and the second apical point marker 314. In an exemplary embodiment, the foreshortening warning 312, 314, 316, 300 may comprise a foreshortening warning message 330. In a representative embodiment, the display system 134 may be configured to present the apical ultrasound image views 310 with at least one apical point marker 312, 314 provided at the automatically detected apical point in the apical ultrasound image views 310 when the amount of movement of the apical point in the apical ultrasound image views 310 over the time period does not exceed the threshold. The at least one apical point marker 312, 314 may indicate one or both of no movement or negligible movement. Alternatively, the display system 134 may be configured to present the apical ultrasound image views 310 without the at least one apical point marker 312, 314 when the amount of movement of the apical point in the apical ultrasound image views 310 over the time period does not exceed the threshold.

Certain embodiments provide a non-transitory computer readable medium having stored thereon, a computer program having at least one code section. The at least one code section is executable by a machine for causing the machine to perform steps 400. The steps 400 may comprise receiving 302 an ultrasound image 200. The steps 400 may comprise receiving 402 apical ultrasound image views 310 over a time period. The steps 400 may comprise automatically detecting 404 an apical point in the apical ultrasound image views 310. The steps 400 may comprise determining 406, 408 an amount of movement of the apical point in the apical ultrasound image views 310 over the time period. The steps 400 may comprise causing 410-416 a display system 134 to present the apical ultrasound image views 310 with a foreshortening warning 312, 314, 316, 330 when the amount of movement of the apical point in the apical ultrasound image views 310 over the time period exceeds a threshold.

In various embodiments, the foreshortening warning 312, 314, 316, 330 may comprise an apical point marker 312 presented at the automatically detected apical point in the apical ultrasound image views 310. The apical point marker 312 may indicate the amount of movement based on color coding in one of a plurality of colors, where each of the plurality of colors corresponds with a different amount of movement. The apical point marker 312 may additionally and/or alternatively indicate the amount of movement based on shading in one of a plurality of shades, where each of the plurality of shades corresponds to the different amount of movement. The apical point marker 312 may additionally and/or alternatively indicate the amount of movement based on marker shape selection in one of a plurality of marker shapes, where each of the plurality of marker shapes corresponds to the different amount of movement. The apical point marker 312 may additionally and/or alternatively indicate the amount of movement based on a label corresponding with the apical point marker, where the label identifies the amount of movement. In an exemplary embodiment, the foreshortening warning 312, 314, 316, 330 may comprise a first apical point marker 312 corresponding with a currently displayed apical ultrasound image view 310 and a second apical point marker 314 corresponding with a previous apical ultrasound image view in the time period. In a representative embodiment, the foreshortening warning 312, 314, 316, 330 may comprise a distance marker 316 corresponding to a distance between the first apical point marker 312 and the second apical point marker 314. In certain embodiments, the foreshortening warning 312, 314, 316, 330 may comprise a foreshortening warning message 330. In various embodiments, the steps 400 may comprise causing 422 the display system 134 to present the apical ultrasound image views 310 with at least one apical point marker 312, 314 provided at the automatically detected apical point in the apical ultrasound image views 310 when the amount of movement of the apical point in the apical ultrasound image views 310 over the time period does not exceed the threshold. The at least one apical point marker 312, 314 may indicate one or both of no movement or negligible movement. The steps 400 may additionally and/or alternatively comprise causing 420 the display system 134 to present the apical ultrasound image views 310 without the at least one apical point marker 312, 314 when the amount of movement of the apical point in the apical ultrasound image views 310 over the time period does not exceed the threshold.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for automatically detecting an apex point in apical ultrasound image views to provide a foreshortening warning.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

1. A method comprising:
acquiring, by an ultrasound system, apical ultrasound image views over a time period;
automatically detecting, by at least one processor, an apical point in the apical ultrasound image views;
determining, by the at least one processor, an amount of movement of the apical point in the apical ultrasound image views over the time period; and
causing, by the at least one processor, a display system to present the apical ultrasound image views with a foreshortening warning when the amount of movement of the apical point in the apical ultrasound image views over the time period exceeds a threshold.

2. The method of claim 1, wherein the foreshortening warning comprises at least one apical point marker presented at the automatically detected apical point in the apical ultrasound image views.

3. The method of claim 2, wherein the at least one apical point marker is one apical point marker indicating the amount of movement based on one or more of:
color coding in one of a plurality of colors, each of the plurality of colors corresponding with a different amount of movement,
shading in one of a plurality of shades, each of the plurality of shades corresponding to the different amount of movement,
marker shape selection in one of a plurality of marker shapes, each of the plurality of marker shapes corresponding to the different amount of movement, and
a label corresponding with the one apical point marker, the label identifying the amount of movement.

4. The method of claim 2, wherein the at least one apical point marker comprises a first apical point marker corresponding with a currently displayed apical ultrasound image view and a second apical point marker corresponding with a previous apical ultrasound image view in the time period.

5. The method of claim 4, wherein the foreshortening warning comprises a distance marker corresponding to a distance between the first apical point marker and the second apical point marker.

6. The method of claim 2, wherein the at least one apical point marker is one of:
colorized pixels of the apical ultrasound image views, or
a marker overlaid on the apical ultrasound image views.

7. The method of claim 1, wherein the foreshortening warning comprises a foreshortening warning message.

8. The method of claim 1, comprising one of:
causing, by the at least one processor, the display system to present the apical ultrasound image views with at least one apical point marker provided at the automatically detected apical point in the apical ultrasound image views when the amount of movement of the apical point in the apical ultrasound image views over the time period does not exceed the threshold, the at least one apical point marker indicating one or both of no movement or negligible movement, or
causing, by the at least one processor, the display system to present the apical ultrasound image views without the at least one apical point marker when the amount of movement of the apical point in the apical ultrasound image views over the time period does not exceed the threshold.

9. A system comprising:
an ultrasound system configured to acquire apical ultrasound image views over a time period;
at least one processor configured to:
automatically detect an apical point in the apical ultrasound image views; and
determine an amount of movement of the apical point in the apical ultrasound image views over the time period; and
a display system configured to present the apical ultrasound image views with a foreshortening warning when the amount of movement of the apical point in the apical ultrasound image views over the time period exceeds a threshold.

10. The system of claim 9, wherein the foreshortening warning comprises an apical point marker presented at the automatically detected apical point in the apical ultrasound image views, the apical point marker indicating the amount of movement based on one or more of:
color coding in one of a plurality of colors, each of the plurality of colors corresponding with a different amount of movement,
shading in one of a plurality of shades, each of the plurality of shades corresponding to the different amount of movement,
marker shape selection in one of a plurality of marker shapes, each of the plurality of marker shapes corresponding to the different amount of movement, and
a label corresponding with the apical point marker, the label identifying the amount of movement.

11. The system of claim 9, wherein the foreshortening warning comprises a first apical point marker corresponding with a currently displayed apical ultrasound image view and a second apical point marker corresponding with a previous apical ultrasound image view in the time period.

12. The system of claim 11, wherein the foreshortening warning comprises a distance marker corresponding to a distance between the first apical point marker and the second apical point marker.

13. The system of claim 9, wherein the foreshortening warning comprises a foreshortening warning message.

14. The system of claim 9, wherein the display system is configured to present one of:
the apical ultrasound image views with at least one apical point marker provided at the automatically detected apical point in the apical ultrasound image views when the amount of movement of the apical point in the apical ultrasound image views over the time period does not exceed the threshold, the at least one apical point marker indicating one or both of no movement or negligible movement, or
the apical ultrasound image views without the at least one apical point marker when the amount of movement of the apical point in the apical ultrasound image views over the time period does not exceed the threshold.

15. A non-transitory computer readable medium having stored thereon, a computer program having at least one code section, the at least one code section being executable by a machine for causing the machine to perform steps comprising:
receiving apical ultrasound image views over a time period;
automatically detecting an apical point in the apical ultrasound image views;
determining an amount of movement of the apical point in the apical ultrasound image views over the time period; and
causing a display system to present the apical ultrasound image views with a foreshortening warning when the amount of movement of the apical point in the apical ultrasound image views over the time period exceeds a threshold.

16. The non-transitory computer readable medium of claim 15, wherein the foreshortening warning comprises an apical point marker presented at the automatically detected apical point in the apical ultrasound image views, the apical point marker indicating the amount of movement based on one or more of:
color coding in one of a plurality of colors, each of the plurality of colors corresponding with a different amount of movement,
shading in one of a plurality of shades, each of the plurality of shades corresponding to the different amount of movement,
marker shape selection in one of a plurality of marker shapes, each of the plurality of marker shapes corresponding to the different amount of movement, and
a label corresponding with the apical point marker, the label identifying the amount of movement.

17. The non-transitory computer readable medium of claim 15, wherein the foreshortening warning comprises a first apical point marker corresponding with a currently displayed apical ultrasound image view and a second apical point marker corresponding with a previous apical ultrasound image view in the time period.

18. The non-transitory computer readable medium of claim 17, wherein the foreshortening warning comprises a distance marker corresponding to a distance between the first apical point marker and the second apical point marker.

19. The non-transitory computer readable medium of claim 15, wherein the foreshortening warning comprises a foreshortening warning message.

20. The non-transitory computer readable medium of claim 15, the steps comprise one of:
causing the display system to present the apical ultrasound image views with at least one apical point marker provided at the automatically detected apical point in the apical ultrasound image views when the amount of movement of the apical point in the apical ultrasound image views over the time period does not exceed the threshold, the at least one apical point marker indicating one or both of no movement or negligible movement, or
causing the display system to present the apical ultrasound image views without the at least one apical point marker when the amount of movement of the apical point in the apical ultrasound image views over the time period does not exceed the threshold.
